(19) United States
(12) Patent Application Publication
(10) Pub. No.: US 2011/0135651 A1
(43) Pub. Date: Jun. 9, 2011

(54) METHOD AND COMPOSITION FOR ENHANCING HEMATOPOIETIC STEM CELL MOBILIZATION

(75) Inventors: Hartmut Geiger, Cincinnati, OH (US); Marnie A. Hall, Mason, OH (US)

(73) Assignee: Children's Hospital Medical Center, Cincinnati, OH (US)

(21) Appl. No.: 13/057,458

(22) PCT Filed: Aug. 18, 2009
(86) PCT No.: PCT/US09/54106

§ 371 Date: Feb. 21, 2011

Related U.S. Application Data
(60) Provisional application No. 61/090,059, filed on Aug. 19, 2008.

(51) Int. Cl. A61K 39/395; A61K 38/18; A61K 31/395; A61K 31/517; A61P 5/00

(52) U.S. Cl. 424/145.1; 514/7.6; 424/158.1; 514/9.6; 514/183; 514/266.4

(57) ABSTRACT

A therapeutic combination for improved mobilization of the hematopoietic stem and progenitor cells, and methods of use thereof are described. The therapeutic combination comprises G-CSF and an inhibitor of the EGFR signaling pathway. The role of EGFR is established by several lines of evidence, including use of quantitative trait locus analysis to map the chromosomal location of the non-G-CSF enhancement of hematopoietic stem and progenitor cells mobilization. Further, several different modes of inhibiting EGFR signaling all provide for an enhanced G-CSF induced mobilization of hematopoietic stem cells.

### TECHNICAL FIELD OF THE INVENTION

The invention relates to the physiology and pharmacological responsiveness of hematopoietic stem and progenitor cells and methods for enhancing mobilization of these cells, and compositions therefor.

### BACKGROUND OF THE INVENTION

Hematopoietic stem and progenitor cells (HSPCs) reside in adults in the bone marrow (BM) and are almost absent from peripheral blood (PB) (Morrison, S I, et al., (1995) Annual Review of Cell & Developmental Biology 11, 35-71). Systemic administration of granulocyte colony-stimulating factor (G-CSF) mobilizes HSPCs from the BM into PB, from which they can be collected for transplantation purposes in clinically useful quantities by leukophoresis (apheresis) (Papayannopoulou, T. et al., (2008) Blood 111, 3923-3930). Mobilization is thus a commonly employed way of harvesting HSPC for stem cell therapies in the clinic. In humans, a 10-fold difference in HSPC mobilization efficiency is observed, and up to 10% of patients fail to mobilize adequate numbers of stem cells for therapeutic purposes upon G-CSF stimulation. Therefore, novel rational approaches for enhancing G-CSF induced HSPC mobilization efficiency are warranted.

Based on a forward genetic quantitative trait locus (QTL) screen in mice the inventors have now determined that the epidermal growth factor receptor (EGFR) acts as a genetic modifier of mobilization efficiency through a Cdc42-mediated pathway. Higher levels of EGFR expression or activity correlated with lower mobilization efficiency, whereas genetic or pharmacological inhibition of EGFR activity during G-CSF treatment significantly enhanced mobilization of HSPC in a “poor” mobilizer mouse strain. Compromised EGFR signaling was associated with lower levels of active Cdc42 whereas EGFR activation by EGF resulted in elevated Cdc42 activity. Subsequent pharmacological inhibition of Cdc42 activity upon G-CSF treatment also enhanced mobilization, implying a causative role for altered Cdc42 activity in regulating the efficiency of G-CSF-induced mobilization downstream of EFGR signaling in vivo. The invention provides a new rationale for targeted pharmacological approaches for individuals who fail to respond adequately to G-CSF-induced mobilization.

### SUMMARY OF THE INVENTION

In view of the need in the art for additional targeted pharmacological regimens for treating a variety of conditions, it is an aspect of the invention to provide a new way to modulate the rate and/or extent of mobilizing various types of stem and progenitor cells. The approach is exemplified herein by describing its application to the enhancement of the effective mobilization of hematopoietic stem and progenitor cells. However, it should be appreciated that the embodied combination of therapeutic agents and its method of use are believed to be generally applicable and, therefore, is not meant to limit the scope of this approach in targeting a specific medical condition in any way.

It is one object of the invention described herein to provide a new combination of therapeutic agents that enhance the mobilization and recruitment of hematopoietic stem and progenitor cells. Generally, this means mobilizing the hematopoietic stem and progenitor cells from surrounding stromal tissue and to stimulate proliferation, and eventually to differentiate into neutrophils. The therapeutic combination comprises compounds from similar or distinct categories of therapeutic agents. Thus, in an example of a two therapeutic combination, each one of the two therapeutic agents can be a different polypeptide, or both can be small molecule pharmaceuticals, and in still another embodiment, the therapeutic combination comprises one polypeptide and one small molecule pharmaceutical. The advantage of the latter combination is that it encompasses a two-pronged approach. Namely, a polypeptide can be expected to affect targets on the external side of the plasma membrane, while a small molecule pharmaceutical will gain access to intracellular targets, thereby expanding the scope of potential therapeutic targets.

Therefore, it is another object of the invention, to mobilize stem cells and/or progenitor cells by treating a subject with a combination of two or more therapeutic agents, and preferably, the two or more therapeutic agents comprise at least one polypeptide and at least one small molecule pharmaceutical.

These objects of the present invention are addressed by providing a combination of therapeutic agents comprising, an effective amount of a therapeutic polypeptide—a hormone, a growth factor, a cytokine, and the like—with a small molecule pharmaceutical. It is contemplated that each of the combination of therapeutics can be co-administered according to any dosing schedule. Thus, the two therapeutic agents can be formulated to be co-administered (i.e., by the same route and at the same time), or to be administered individually but simultaneously or concomitantly (i.e., by different routes but at the same time or in an overlapping time period), or by different routes at distinct, i.e., non-overlapping time periods of administration.

It is yet another object of the invention to demonstrate the mobilization of hematopoietic stem and progenitor cells, by administering a combination comprising, an effective amount of G-CSF (granulocyte colony-stimulating factor), and an effective amount of at least one inhibitor of EGFR (epidermal growth factor receptor), or an inhibitor of a cellular target stimulated by the activated EGFR.

It is yet another object of the invention to provide a combination of therapeutic agents comprising an amount of AMD3100 effective to enhance mobilization of HSPC, and an amount of at least one inhibitor of EGFR effective to inhibit the effects of EGFR signaling.

It is still yet another object of the invention to provide a method to enhance G-CSF-induced mobilization of hematopoietic stem and progenitor cells in a subject in need thereof. This method comprising administering a therapeutic combination comprising an amount of AMD3100 effective to enhance mobilization of HSPC, and an amount of at least one inhibitor of EGFR effective to inhibit EGFR signaling.

### BRIEF DESCRIPTION OF THE DRAWINGS

FIG. 1. Identification of a genomic region (locus) that contains a gene that regulates HSPC mobilization. (a) Genetic constitution of B6.D2 chr11 (line G) described on page 11, first paragraph and novel sub-congenic lines generated from line G (B., C57BL/6 allele, D., DBA allele). Square represents the 5 Mbp interval. (b) Frequency of CFC per 37.5 μl of PB post G-CSF induced mobilization in B6 (n=10) and line G (n=10) (chr.11 0-36 Mbp) mice. *p<0.05 versus B6. (c) Mobilization efficiency of subcongenic lines 106 (n=4), 338 (n=7), 1023 (n=4) and 1804 (n=8) compared to B6 and line G. *p<0.05 versus B6, #p<0.05 versus line G. Error bars represent the mean±s.e.m. Note that the results are normalized to the efficiency of line B6., i.e., B in the figure. Thus regulation of mobilization efficiency is linked to a 5 Mbp interval on murine chromosome 11.

FIG. 2. Relative differences in expression in HSPCs (LIN−, ckit+ cells) from BM of the genes in the 5 Mbp interval represented on the MOE430 chip, with the level of expression for C57BL/6 set to 1. Data is based on three independent hybridizations per genotype. Both probe sets for EGFR show lower level of expression in line G, which could be subsequently confirmed by RT-PCR. p<0.05.

FIG. 3. EGF reduced mobilization efficiency of HSPC. (a) EGFR expression by real time Q-RT-PCR in bone marrow derived HSPC (LIN−, ckit+) from B6 and line G animals before and after treatment with G-CSF to induce mobilization (n=3 repeats per experimental group)*p<0.05 versus B6 at steady state or mobilized, #p<0.05 versus line G at steady state. (b) Mobilization efficiency in 136 (n=6) mice after a single dose of EGF on day 5 of the standard G-CSF regimen G., G-CSF, E., EGF. Mobilization was determined approximately 16 hours post-EGF injection. (c) 136 mice mobilized with G-CSF and treated with EGF (0.8 mg/g) on day 5. (d) wa2 mice mobilized with G-CSF and treated with EGF (0.8 mg/g) on day 5. (e) Schematic of the setup for competitive transplant experiments to measure stem cell frequency in PB using identical volumes of PB as donor tissue from mice mobilized by either G-CSF (n=3) or G-CSF plus EGF (n=4) for the 2 experimental groups. (f) Relative donor chimerism in PB 3 months post transplant in recipient animals competitively transplanted with identical volumes of PB from mice mobilized with either G-CSF or G-CSF plus EGF in competition to identical numbers of B6.CD45.1 BM cells as determined by flow cytometry. (g) Mobilization efficiency of line 1804 compared to B6 after G-CSF and EGF treatment. *p<0.05 versus G-CSF. Error bars represent the mean±s.d.

FIG. 4. Expression of EGFR ligands in hematopoietic cells obtained from total bone marrow (TBM). PCR was performed using specific primers (see Table 1, below) for EGF (epidermal growth factor), including TGF-α (transforming growth factor-α), HB-EGF (Heparin-binding EGF-like growth factor) and BTC (betacellulin), using cDNA isolated from low density bone marrow.

FIG. 5. Genetic and pharmacologic inhibition of EGFR activity enhances mobilization efficiency. (a) Schematic of the experimental setup of the transplants to determine mobilization efficiency of wa2 hematopoietic cells (b) Mobilization efficiency (CFCs per 37.5 μl of PB) in control and wa2 recipient mice (n=6/group) mobilized by the standard G-CSF. *P<0.05 versus wt. (c) Schematic of the experimental setup for competitive transplant experiments to determine whether increased mobilization efficiency is intrinsic to wa2 progenitor cells. (d) Relative progenitor cell mobilization efficiency as determined by the chimerism in the progenitor compartment (lin−, C-kit+) in PB post standard G-CSF mobilization relative to chimerism of hematopoietic cells in PB before mobilization. (e) Mobilization efficiency (number of CFCs per 37.5 μl of PB) in response to G-CSF or G-CSF plus Erlotinib (2.5-10.0 mg/kg) (on day 3, 4 and 5 of the G-CSF regimen) treatment. (f) Schematic of the set-up for competitive transplant experiments to measure stem cell frequency in PB using as donor tissue identical volumes of PB from mice mobilized by either G-CSF or G-CSF plus Erlotinib for the 2 experimental groups (3 recipients/group). (g) Relative donor chimerism in PB 3 months post transplant in recipient animals competitively transplanted with identical volumes of PB from mice mobilized with G-CSF or G-CSF plus EGF in competition to identical numbers of B6.CD45.1 BM cells as determined by flow cytometry. *P<0.05 versus G-CSF. Error bars represent the mean±s.d.

FIG. 6 is a graphical representation of the effect a treatment combining Erlotinib and AMD3100 has on mobilization as compared to a treatment with AMD3100 alone.

FIG. 7. Cdc42 regulates mobilization efficiency in response to EGFR signaling. (a) Quantification of progenitor cell adhesion to a FBMD-1 stromal layer after either G-CSF or G-CSF plus EGF (200 ng/ml) treatment. (b) Quantification of adhesion of progenitor cells to FBMD-1 stromal cells after either G-CSF or G-CSF plus Erlotinib (10 mM) treatment (data represents at least 3 individual experiments). (c) Representative immunoblot demonstrating increase of Cdc42 activity in vivo in LD BM cells from G-CSF mobilized B6 animals upon EGF treatment (n=2 separate experiments) (d) Representative immunoblot demonstrating decrease of Cdc42 activity in vivo in LD BM cells from G-CSF mobilized B6 animals in vivo in response to Erlotinib (n=2 separate experiments). (e) Cdc42 activity in LD BM cells in response to G-CSF mobilization in “poor mobilizer” B6 and “better mobilizer” line 1804 (representative of 2 individual experiments with 3 mice/group). *=P<0.05. Error bars represent the mean±s.d.

FIG. 8 is a graphical representation of EGFR expression in human hematopoietic progenitor cells.

### DETAILED DESCRIPTION OF THE INVENTION

As used in the context of the present invention, the term “hematopoietic stem and progenitor cells,” i.e., HSPC, are self-renewing precursors that regenerate myeloid and lymphoid cells throughout the life span of the subject or patient. The term “stem cell” is meant to encompass stem cells and progenitor cells of various levels of pluripotency.

The terms “subject” and “patient” are used interchangeably for the purpose of this description, wherein either a subject or a patient refers to a living mammal, which includes humans and other mammals that persons of ordinary skill in the art commonly use.

In the context of the present invention, the term “mobilization” of hematopoietic stem and progenitor cells, i.e., HSPC, refers to the recruitment of HSPC into the blood. Generally, HSPC are found in bone marrow, spleen, umbilical cord blood, and the blood and liver of fetuses and newborns. Generally, cells obtained from bone marrow, cord blood, or mobilized peripheral blood of healthy donors, are clinically useful for transplantation into a recipient subject.

In one embodiment, the method of the present invention is directed to enhancing the mobilization of HSPC beyond the level expected by treating a subject with a cytokine, preferably a cytokine with colony-stimulating and cell differentiation-inducing properties. In the nonlimiting example illustrated herein, the cytokine is G-CSF. The preferred manner to enhance HSPC mobilization over the level achieved by administering only G-CSF, is to provide an additional therapeutic agent to be administered with G-CSF. It is noted that the term “with G-CSF” does not require simultaneous or even overlapping co-administration of G-CSF and the second therapeutic agent. More accurately, the “with G-CSF” refers to any suitable treatment regimen wherein G-CSF and an additional therapeutic agent combine to effectively enhance the level of HSPC mobilization over that seen with G-CSF alone.

One pathway by which G-CSF seems to induce stem cell mobilization is by the disruption of the interaction of the chemokine stromal derived factor 1 (SDF-1) with its receptor CXCR4 located on stem cells. AMD3100 is one selective antagonist of the CXCR4 receptor and disrupts the binding of SDF-1 to CXCR4, resulting in mobilization of HSPC's. 1,1′-[1,4-phenylene-bis(methylene)]-bis-1,4,8,11-tetraazacyclotetradecane-, commonly referred to as plerixafor or AMD3100 is described more fully in U.S. Pat. No. 5,583,131, which is incorporated herein by reference. AMD 3100 is marketed as a component of Mozobil® by the Genzyme Corporation. Generally, AMD 3100 is an antagonist with the CXCR4 chemokine and interferes with the binding of SDF-1 with CXCR4 on stem cells which leads to the release of HSPC from bone marrow into PB.

The EGFR is activated by several ligands in addition to EGF (epidermal growth factor), including but not limited to TGF-α (transforming growth factor-α), HB-EGF (Heparin-binding EGF-like growth factor), BTC (betacellulin), amphiregulin and epiregulin. However, compounds are also known that directly or indirectly inhibit the effects of EGFR activation. The direct inhibitors interfere with the functioning of polypeptide domains on EGFR itself. In contrast, the indirect inhibitors interfere with the functioning of the downstream molecular targets that are themselves, activated by EGFR binding by one of the above-mentioned activating ligands. The method of the present invention as described herein illustrates that administering G-CSF with an inhibitor of the EGFR and/or any of the EGFR's downstream intracellular effectors significantly enhances HSPC mobilization. Therefore, in one embodiment, the G-CSF is administered with an additional polypeptide capable of inhibiting the EGFR's intracellular signaling function. The additional polypeptide may be an antibody—monoclonal, polyclonal or various engineered or chemically modified active fragments thereof—that prevent, inter alia, binding of EGF to the EGFR. The method further contemplates analogues of EGF that interfere with normal EGF binding to the EGFR. An additional embodiment may comprise administering G-CSF with an anti-EGF antibody, or engineered or chemically modified variant, that would effectively diminish blood EGF levels below a physiologically effective level.

It is further contemplated that G-CSF can be administered with one or more small molecule pharmaceuticals that inhibit the effects of the EGFR signaling. Small conventional pharmaceuticals can rapidly enter cells and effect intracellular targets in ways that polypeptide factors cannot. In view of this difference in cellular targets, it is further contemplated that G-CSF may be co-administered with a combination of anti-EGFR compounds; i.e., a cocktail, comprising any of the anti-EGFR polypeptide factors described above in combination with a small molecule pharmaceutical, such as various classes of protein kinase inhibitors.

Thus, it is further contemplated that co-administering one or more compounds that can selectively inhibit the EGFR tyrosine kinase can enhance the efficiency of G-CSF on HSPC mobilization. In addition, the use of specific inhibitors known to inhibit the downstream effector protein kinase, e.g., cdc42, is also encompassed by the described invention.

Therefore, persons of ordinary skill in the art will appreciate that this approach can be modified as the list of inhibitors of EGFR and its downstream effectors continue to increase in number. Accordingly, the following illustrated examples are not meant to limit the scope of the invention.

### Example 1

### Chromosomal Mapping of Effectors HSPC Mobilization Efficiency

Therapeutic Agent Regimen.

As mobilization efficiency of HSPC is a quantitative trait, quantitative trait locus (QTL) analysis was chosen as an approach to identify regulators of mobilization efficiency. Mice were mobilized with huG-CSF (Amgen) at 12.5 μg/ml in PBS/0.1% BSA and administered i.p. at 100 μg/kg/day once a day for 5 days and animals were analyzed on day 6. Murine recombinant EGF (0.2-3.6 μg/g) (Prepro tech, Rocky Hill, N.J.) was dissolved in PBS and administered i.p. on the last day of the G-CSF regimen. Erlotinib (2.5-100 mg/kg) was dissolved in methylcellulose and administered by gavage on day 3, 4, and 5 of the G-CSF regimen. A specific Cdc42 inhibitor (0.5 mg/ml) was dissolved in PBS containing 15% ethanol and administered by tail vain 18 h after the last G-CSF injection. Mobilization efficiency was determined with HSPC obtained approximately 14-16 hours after the last injection received by the group of mice.

Animals. C57BL/6 mice (6-8 weeks) were obtained from NCI and subsequently housed in the animal barrier facility at Cincinnati Children's Hospital Medical Center (CCHMC). B6.SJL(BoyJ) mice were either obtained from the divisional stock (derived from animals obtained from The Jackson Laboratory) or obtained from NCI (C57BL/6 Ly5.2Cr). Waved-2 (wag) animals were obtained from Nancy Ratner and housed in the animal barrier facility at CCHMC.

Colony forming cell assays. 150 μl of peripheral blood (PB) was added to Hank's balanced salt solution (HBSS) and mixed with 4 ml of methylcellulose (Stem Cell Technologies) containing 50 ng/ml rm SCF, 10 ng/ml rm IL-3 and 10 ng/ml rh JL-6 and incubated at 37° C. Samples were plated in triplicate in 6 well plates (Falcon) and colonies of more than 50 cells were counted between 7 to 10 days. CFC counts were also determined in spleen (1×10⁵) using the same protocol. The results are presented as mean±s.d. A paired student's t-test was used to determine the significance of the difference between means of two groups. Values were considered significant when p<0.05.

Whole genome expression analysis. RNA from sorted Lin-C-Kit+ cells were obtained with the Qiagen RNA assay micro kit according to the protocol of the manufacturer. RNA was subsequently linear amplified by the Affymetrix core at CCHMC and reverse transcribed with a Nugene Kit according to the manufacturer protocol. Labeled cDNA was then hybridized to and MOE430 array (Affymetrix) and raw expression data collected. Affymetrix .CEL files of the respective microarrays were imported into the statistical programming language R (www.r-project.org) using the affy Bioconductor (www.bioconductor.org) package. The data were then pre-processed (log2-transformed, background corrected, quantile normalized and summarized) using the rma function of the affy package. Affymetrix probes were filtered and re-grouped during summarization according to RefSeq annotation using custom chip description files (.CDF) for the MOE430 array provided by the Molecular and Behavioral Neuroscience Institute of the University of Michigan (Microarray Lab), http://brainarray.mbni.med.umich.edu/Brainarray/Database/CustomCDF/genomic_curated_CDF.asp). The expression level of the EGFR was subsequently confirmed by a Taqman real time PCR assay kit from Applied Biosystems (Assay ID: Mm01187863_g1)

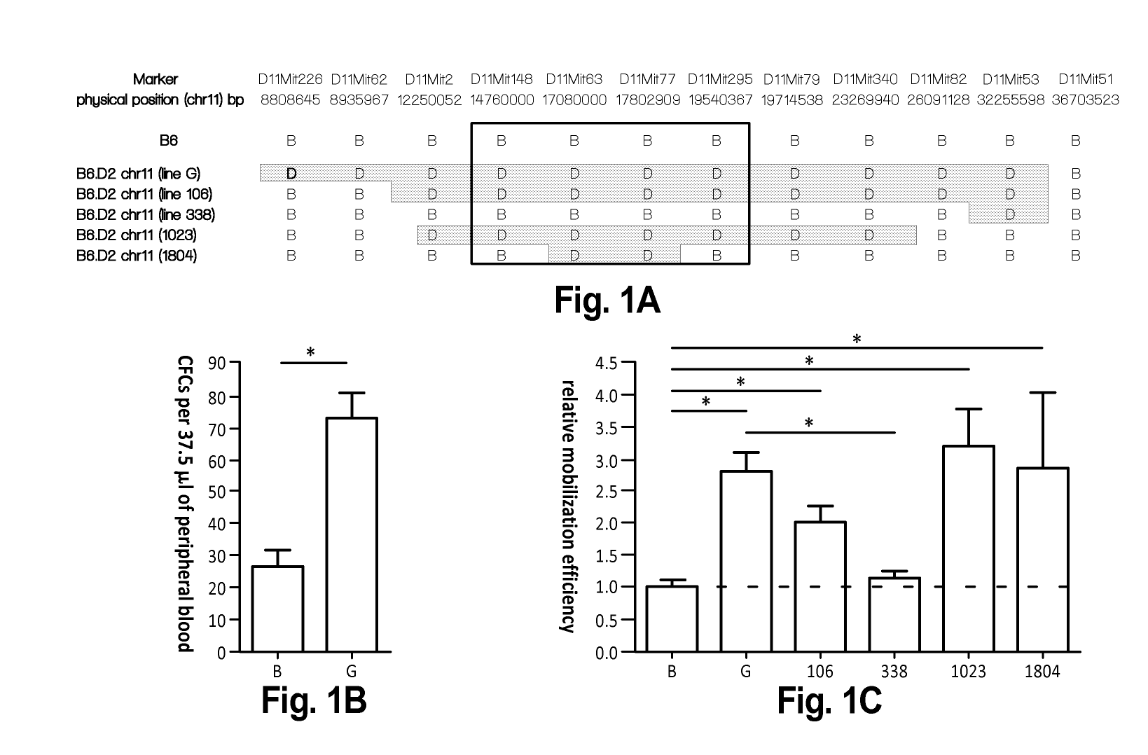

A locus on murine chromosome 11 (0 to 36 Mbp) that participated in regulating HSPC mobilization efficiency was previously demonstrated by generating congenic strains and screening them for such linkages effecting mobilization efficiency. It was concluded that a DBA/2 allele in the congenic line B6.D2 chr.11 (0-36 Mbp, line G) conferred an approximately 2-fold increase in mobilization efficiency (FIG. 1b).

To further narrow the interval, novel congenic animals were generated from the originally described line G by further backcrossing congenic animals to B6 mice and utilizing marker-assisted selection of offspring that bear a novel cross-over in the interval encompassing 0-36 Mbps (FIG. 1a). Mobilization efficiency of the parental as well as the novel congenic strains (FIG. 1c) was determined using the standard G-CSF mobilization protocol. Novel congenic lines 106 (D2 interval 8.9 to 36.7 Mbp), 1023 (D2 interval 8.9 to 26.1 Mbp) and 1804 (D2 interval 14.7 to 19.5 Mbp) showed a significant increase in mobilization efficiency compared to 36 mice, whereas line 338 (D2 interval 26.1 to 36.7 Mbp) presented with a B6 phenotype (FIG. 1e). Thus, the putative interval conferring enhanced mobilization efficiency of stem and progenitor cells was dramatically narrowed to the interval between 14.7 to 19.5 Mbp on murine chromosome 11. This region is less than 5 Mbp and less that 14% of the originally mapped 36 Mbp starting interval.

Twelve transcripts are located within the 5 Mbp on murine chromosome 11 (FIG. 1a), including the epidermal growth factor receptor (EGFR), a cell surface receptor with tyrosine kinase activity (RTK). RTKs (like c-Kit) are known to play a role in the regulation of stem cell localization and therefore EGFR was a regarded as a potential candidate gene. At present, there is no consensus in the art as to whether the EGFR is expressed in hematopoietic cells. To first determine EGFR expression in hematopoietic cells quantitative real-time PCR for EGFR cDNA was performed (Table 1).

TABLE 1
Expression of EGFR

| | EGFR/actin | SEM |
|---|---|---|
| Hemotopoietic progenitor cells | 0.023 | 0.002 |
| Peripheral blood | 0.153 | 0.004 |
| Lung | 34.287 | 1.547 |
| Brain | 48.432 | 4.937 |
| Liver | 1713.590 | 61.413 |

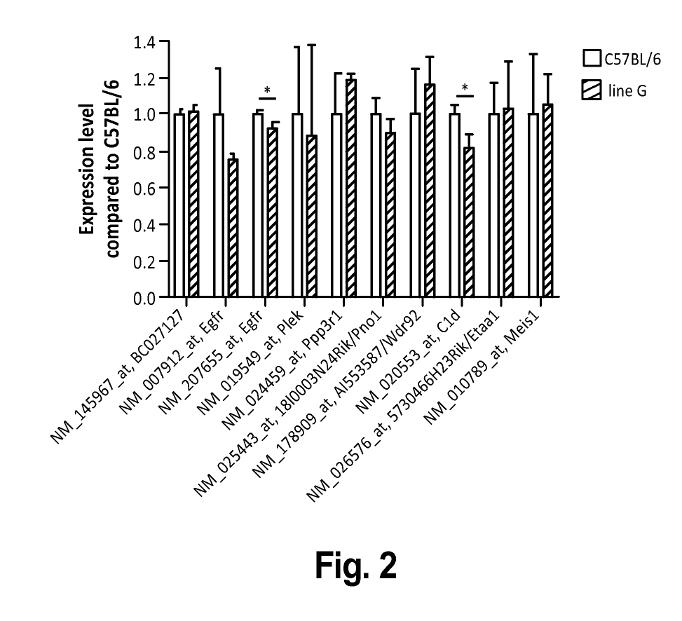

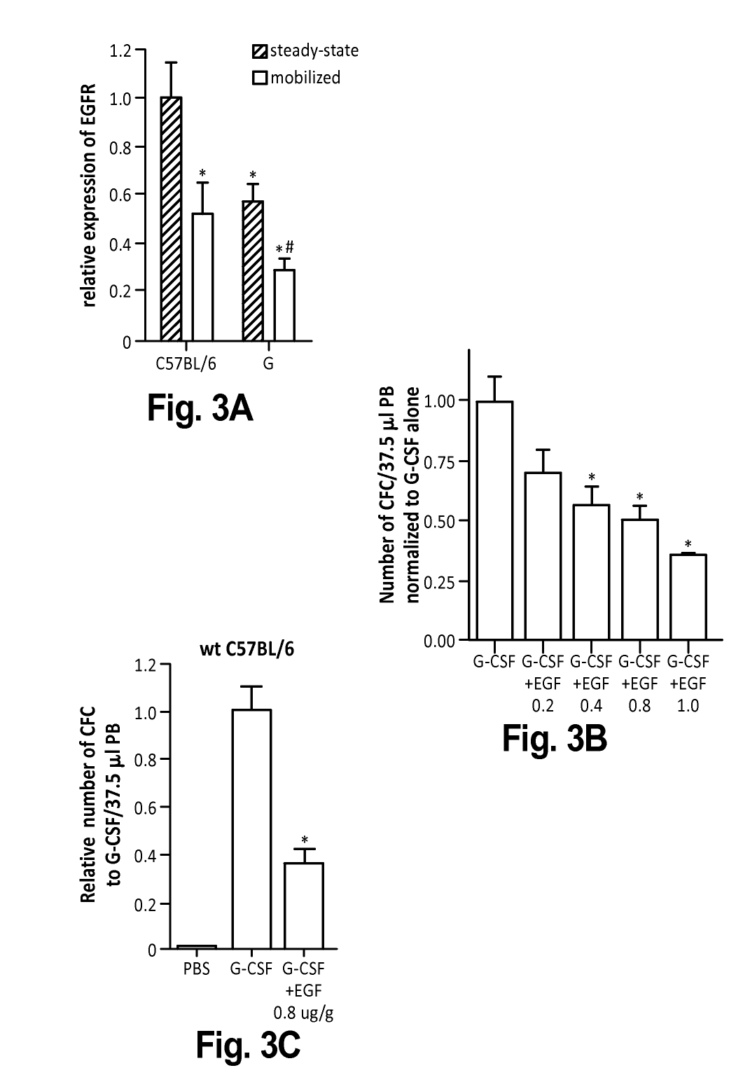

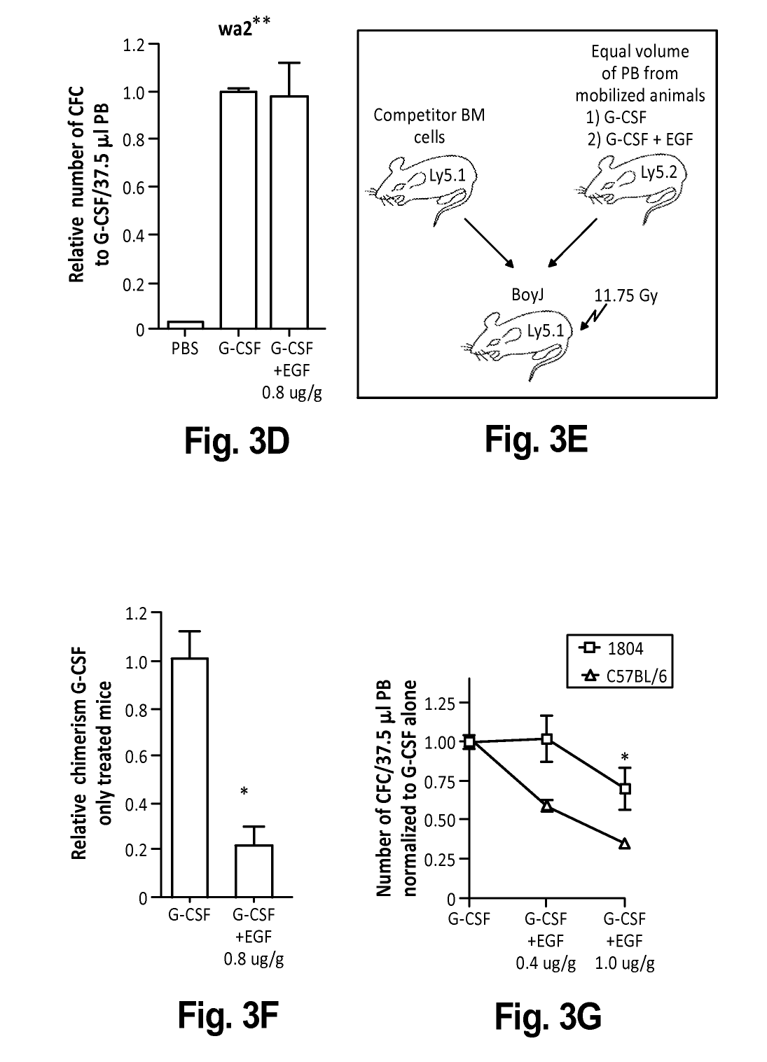

Expression of EGFR transcripts was detected in both bone marrow and progenitor cells (LIN−, c-Kit+), albeit at a very low level. Whole genome expression analyses further suggested the EGFR to be one out of 2 genes located in the 5 Mbp interval to present with differential expression between the congenic strain and the control B6 strain (FIG. 2). Significant differential expression of the EGFR in progenitor cells from the congenic strain (line G) (high mobilizer) and C57BL/6 mice (low mobilizer) could be confirmed in both steady state (unstimulated) and G-CSF treated animals (mobilized) (FIG. 3a). EGFR expression levels in BM HSPCs decreased upon G-CSF treatment and were inversely correlated to mobilization efficiency in G-CSF stimulated animal, suggesting a negative role for EGFR signaling in regulating mobilization efficiency.

### Example 2

### EGFR-Mediated Reduction of HSPC Mobilization Efficiency

Transplantations/Competitive transplantations. BM cells were harvested and pooled from the tibia and the femur of 6-8 week old mice (donor) as well as B6.5JL(BoyJ) (competitor) mice. Equal numbers of BM cells (2×10⁶ cells of each competitor and donor) were transplanted into BoyJ (recipients) mice that were lethally irradiated with a total dosage of 11.75 Gy (7 Gy+4.75 Gy, 4 hours apart). BM cells were subsequently transplanted into the retro-orbital sinus in a volume of 200 μA in IMDM/2% FCS.

Flow cytometry. Immunostaining and flow cytometry analyses were performed according to standard procedures and analyzed on a FacsCanto flow cytometer (BD Biosciences). Anti-Ly5.2 (clone 104, BD Biosciences, FITC conjugated) and anti-Ly5.1 (clone A20, BD Biosciences, PE conjugated) monoclonal antibodies were used to distinguish donor from recipient and competitor cells. For lineage analysis in hematopoietic tissues, anti-CD3ε (clone 145-2C11, PE-Cy7 conjugated), anti-B220 (clone RA3-6B2, APC conjugated), anti-CD11b (clone M1/70, APC-Cy7 conjugated) and anti-Gr-1 (clone RB6-8C5, APC-Cy7 conjugated, all from BD Biosciences) were used.

To test our hypothesis on a possible inhibitory role of EGFR signaling on mobilization efficiency, mice were mobilized with G-CSF and administered a single dose of murine recombinant EGF (0.2-1.0 twig) on the last injection day of the G-CSF regimen. Results demonstrated a dose-dependent inhibition of mobilization efficiency of progenitor cells by EGF in G-CSF stimulated animals (approx. 4-fold reduction using 0.8 μg/g) (FIG. 3b) while EGF at the dose range tested did not restrict spontaneous mobilization in non-G-CSF treated animals (data not shown). To determine if the inhibition of mobilization by EGF is dependent upon the activity of the EGFR, we utilized the waved2 (wa2) mouse, a strain that bears a naturally occurring T to G transversion mutation in the sequence encoding the tyrosine kinase domain of the EGFR leading to a reduction in receptor activity to about 10%. Both wt and wa2^{+/-} mice were mobilized with either G-CSF alone or G-CSF plus a single injection of EGF on day 5. Mobilization efficiency of wild type mice was significantly reduced with treatment with EGF (FIG. 3c) while wa2^{+/-} mice were unaffected by EGF (FIG. 3d) demonstrating that the distinct level of EGFR activity is necessary for EGF to inhibit progenitor cell mobilization efficiency.

To test the level of inhibition on mobilization of stem cells post EGF treatment, competitive transplantations were performed using equal volumes of blood from both G-CSF and G-CSF plus EGF (0.8 μg/g) treated mice (FIG. 3e). Animals treated with G-CSF plus EGF contributed significantly less to chimerism 3 months post transplant (approx. 5-fold) compared to animals transplanted with G-CSF alone and thus mobilized up to 5-fold less stem cells upon EGF treatment to PB (FIG. 3f). Consistent with our analyses so far, the congenic line 1804, bearing the D2 allele of the EGFR which results in reduced expression was significantly less sensitive to inhibition of mobilization efficiency by EGF compared to B6 mice (FIG. 3g).

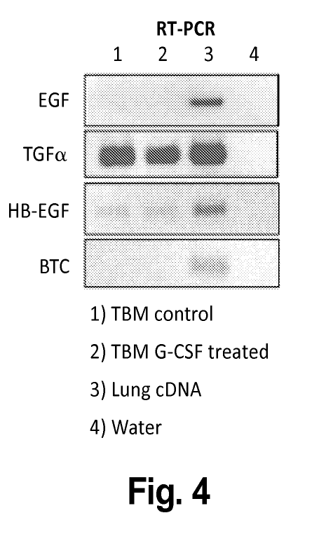

FIG. 4. illustrates the expression levels of EGFR-activating ligands in HSPC. The EGFR is activated by several ligands in addition to EGF (epidermal growth factor), including but not limited to TGF-α (transforming growth factor-α), HB-EGF (Heparin-binding EGF-like growth factor) and BTC (betacellulin), amphiregulin and epiregulin. Expression of both TGF-α and HB-EGF, but not EGF and BTC were detected by RT-PCR in bone marrow cells, demonstrating the presence of known activators of the EGFR in G-CSF treated animals in bone marrow. See Table 2 for sequences of primers used in the PCR reactions. Taken together, these data suggests that EGFR signaling in vivo might alter mobilization efficiency and this pathway might, at least in part, regulate inter-strain differences in mobilization efficiency.

TABLE 2
Primer Sequences

| | | | Prod- |
|---|---|---|---|
| | | | uct |
| | | Tm | size |
| Gene | Sequence | (° C.) | (bp) |
| Egf | Sense: 5′-GAGAGGTGCAG | 57.3 | 271 |
| | GACCTG-3′ (SEQ ID NO: 1) | | |
| | Anti-Sense: 5′-CACCAATTGC | 52.6 | |
| | TGGTGATTTG-3′ | | |
| | (SEQ ID NO: 2) | | |
| Betacellulin | Sense: 5′-GGAACCTGAGGA | 56.1 | 181 |
| (BTC) | CTCATCCA-3′ (SEQ ID NO: 3) | | |
| | Anti-Sense: 5′-GAGCCATTGGT | 55.6 | |
| | TTCTGGTGT-3′ | | |
| | (SEQ ID NO: 4) | | |
| TGF-□ | Sense: 5′-TGTGTGATAAAG | 55.8 | 100 |
| | CTGCCTGC-3′ (SEQ ID NO: 5) | | |
| | Anti-Sense: 5′-CAACCCTTTGA | 55.4 | |
| | GGTTCGTGT-3′ | | |
| | (SEQ ID NO: 6) | | |
| HB-Egf | Sense: 5′-ATAGCTTTGCG | 57.1 | 166 |
| | CTGTGACCT-3′ | | |
| | (SEQ ID NO: 7) | | |
| | Anti-Sense: 5′-CACACTCTTT | 57.1 | |
| | GGTCCCACCT-3′ | | |
| | (SEQ ID NO: 8) | | |

### Example 3

### Mechanism(s) of EGFR Modulation of HSPC Mobilization

CAFC progenitor adhesion assays. FBMD-1 cells were seeded in IMDM supplemented with 15% FCS and 5% horse serum (Gibco) at a density of 1000 cells per well in a 96 well plate. BM cells were plated onto the FBMD-1 stroma cell line at 3000, 1500, 750 and 375 cells per well at 15 wells per cell concentration in CAFC medium (IMDM, supplemented with 20% horse serum (Gibco) and 10⁻⁵ M hydrocortisone (Sigma)). To determine progenitor cell adhesion, non-adherent cells were washed off the FBMD-1 stroma after 2 hours and fresh CAFC medium was added to each well. The frequency of total HSPCs and adherent HSPCs was determined by counting the frequency of cobblestone areas at day 7 on the FBMD-1 stroma cell line.

Rho-GTPase effector domain pull-down assays. Relative levels of GTP-bound RAC1, RAC2 and CDC42 were determined by an effector pull-down assay. Briefly, low density bone marrow cells (1×107) were lysed in a Mg2+ lysis/wash buffer (Upstate cell signaling solutions) containing 10% glycerol, 25 mM sodium fluoride, 1 mM sodium orthovanadate and a protease inhibitor cocktail (Roche Diagnostics). Samples were incubated with PAK-1 binding domain/agarose beads and bound (activated) as well as unbound (non-activated) Rho GTPases were probed by immunoblotting with antibodies specific for RAC1 (Upstate), RAC2 (Novus Biologicals) and CDC42 (BD Biosciences). Activated protein was normalized to total protein and β-actin (Sigma) and the relative amount was quantified by densitometry.

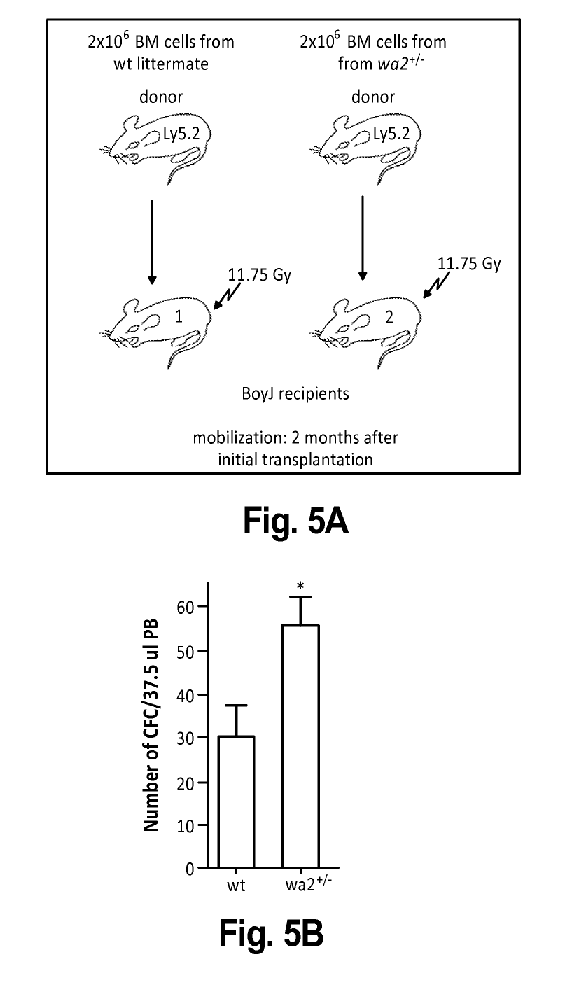

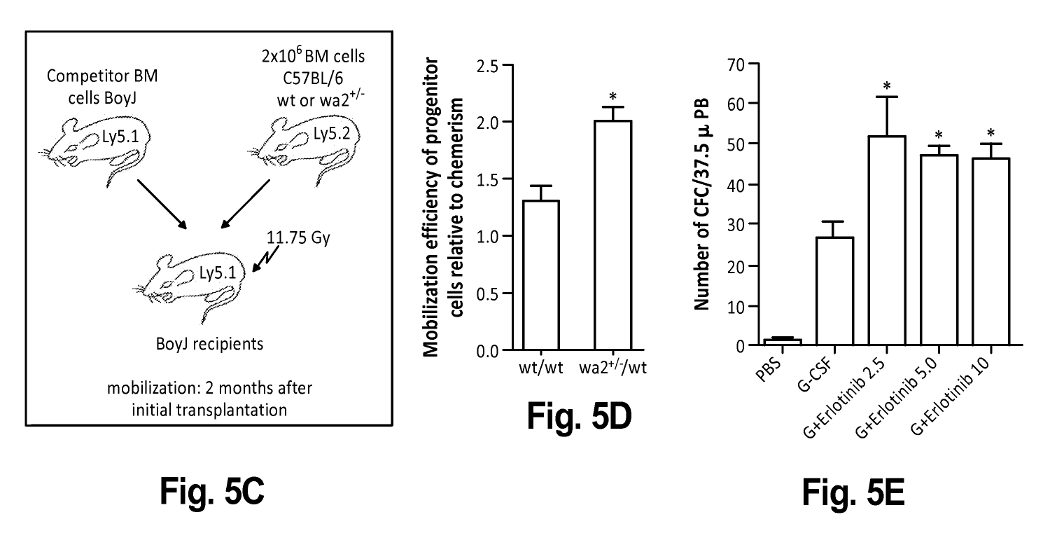

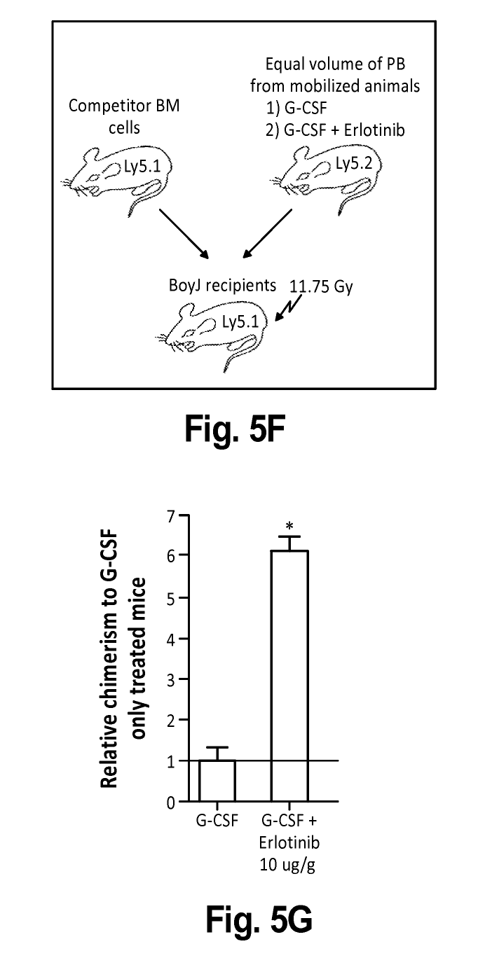

The overall goal of our studies is, via a genetic approach, to identify therapeutic targets to increase mobilization efficiency. Based on our data presented so far, activation of the EGFR pathway reduces mobilization efficiency of stem and progenitor cells which leads to the conclusion that inhibition of EGFR expression/signaling might improve mobilization in the poor mobilizer strain 86. To test this novel hypothesis, both a genetic and pharmacological approach was taken. To investigate the role of reduced EGFR signaling in hematopoiteic cells with respect to mobilization efficiency, animals in which the hematopoietic system was reconstituted with littermate control or wa2^{+/-} hematopoietic cells underwent G-CSF induced mobilization (FIG. 5a). Animals reconstituted with wa2^{+/-} hematopoietic cells presented with a significant increase in the number of progenitor cells mobilized to PB compared to animals reconstituted with BM cells from littermate control animals (FIG. 5b). To determine if the increase in mobilization efficiency due to reduced EGFR activity is progenitor cell intrinsic, competitive transplantations were performed using BM cells from either wa2^{+/-} or wt mice (Ly5.2) and competitor BM cells (Ly5.1) (FIG. 5c). In recipients competitively transplanted with wa2^{+/-} BM cells significantly higher frequencies of wa2^{+/-} progenitor cells were mobilized relative to their chimerism before mobilization, demonstrating that the role of EGFR signaling in mobilization is mostly intrinsic to progenitor cells (FIG. 5d).

To further test whether pharmacological inhibition of EGFR activity results in enhanced mobilization efficiency, mice were mobilized with G-CSF and treated with Erlotinib, which specifically inhibits EGFR activity Treatment with Erlotinib over a course of the last 3 days of the G-CSF regimen at a dose of 2.5-10 mg/kg significantly increased mobilization efficiency of progenitor cells (FIG. 5e) and stem cells (measured again by using competitive transplantation experiments as a read-out for stem cell frequency) (FIG. 5f, 3g). Thus, targeting EGFR activity by Erlotinib has been identified as a novel therapeutic approach for increasing G-CSF induced stem cell mobilization efficiency in a poor mobilizer mouse strain. In summary, reduced activity of the EGFR (either by genetic or pharmacological means) results in enhanced G-CSF induced mobilization efficiency.

Further, inhibition of EGFR signaling by administering a combination of Erlotinib and AMD3100 enhances mobilization as compared to mobilization resulting from administration of AMD3100 alone. To collect mobilized HSPC's through apheresis after an administration of AMD3100 alone, one must wait several hours for a sufficient quantity of HSPC's to mobilize to the PB. In one embodiment, administering a combination of Erlotinib and AMD3100 results in a mobilization of HSPC's from bone marrow to PB, allowing for apheresis between about 30 minutes and about 24 hours after administration. More preferably, administering a combination of Erlotinib and AMD3100 results in a mobilization of HSPC's from bone marrow to PB, allowing for apheresis between about 2 hours and about 10 hours after administration.

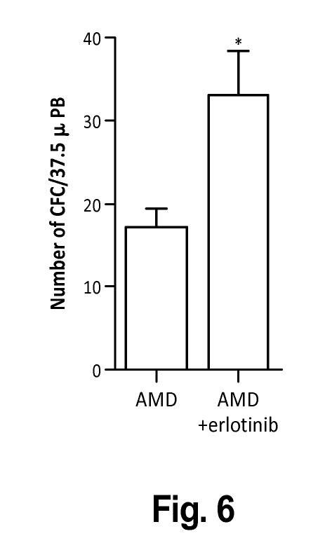

This decrease in time between administration and apheresis of the combination of Erlotinib and AMD3100 as compared to AMD3100 alone can be seen in FIG. 6. FIG. 6 shows an approximately 100% increase in mobilization efficiency (CFCs per 37.5 it of PB) when a subject is treated with a combination of Erlotinib and AMD3100 as compared to treatment with AMD3100 alone.

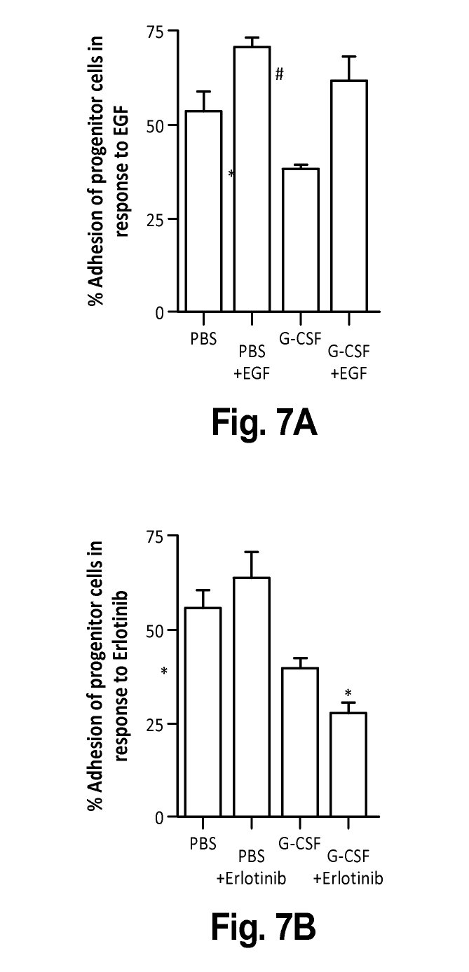

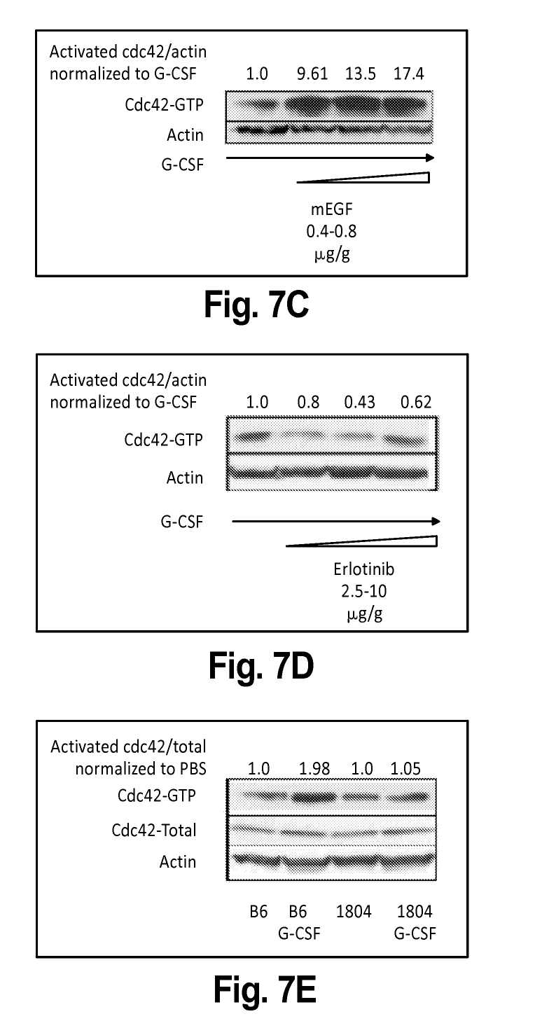

To identify possible cellular and molecular mechanisms by which EGF receptor signaling alters mobilization efficiency, cell adhesion assays were performed as well as the activity/expression of known downstream targets of EGFR signaling were investigated. Since de-adhesion of cells from the stroma is a pre-requisite for mobilization ((Papayannopoulou, T. et al., (2008) Blood 111, 3923-3930), the ability of BM derived progenitor cells from G-CSF treated animals to adhere to stroma in response to EGF or Erlotinib treatment was determined by a CAFC adhesion assay (Xing, Z., et al. (2006) Blood 108, 2190-2197). Interestingly and consistent with our hypothesis, EGF treatment of BM derived HSPCs resulted in significantly enhanced adhesion of progenitor cells to stroma, (FIG. 7a) whereas treatment with Erlotinb resulted in significantly reduced adhesion (FIG. 7b). Thus EGFR signaling apparently regulates mobilization efficiency via altering cell adhesion.

Known prominent targets of EGFR signaling include the family of small Rho GTPases Rac1, Rac2 and Cdc42. Changes in the activity of these proteins have previously been shown to play an important role in both migration and adhesion of stem and progenitor cells. (Yang, F. C., (2001). et al. Proc Nati Acad Sci USA 98, 5614-5618); Cancelas, J. A., (2005). et al. Nat Med 11, 886-891; Yang, L., (2007). et al. Proc Nati Acad Sci USA 104, 5091-5096).

Effector-domain GST fusion pull down experiments on bone marrow cells were performed to determine whether activation of EGFR signaling in hematopoietic cells in vivo affected the activity of Rac1, Rac2 or Cdc42. Consistent with the literature on fibroblasts, activation of EGFR signaling by EGF in vivo in G-CSF treated animals resulted in a significant increase in Cdc42 activity in BM cells (FIG. 7c) compared to G-CSF treatment alone. Conversely, inhibition of EGFR signaling by Erlotinib resulted in a significant decrease in the GTP-bound form of Cdc42 compared to G-CSF alone treated animals (FIG. 7d). We did not detect a significant change in the level of the GTP bound forms of Rac1 or Rac2 (data not shown). Collectively these data demonstrate that changes in EGFR signaling upon G-CSF induced mobilization affect the active level of Cdc42, which in turn inversely correlates with mobilization efficiency.

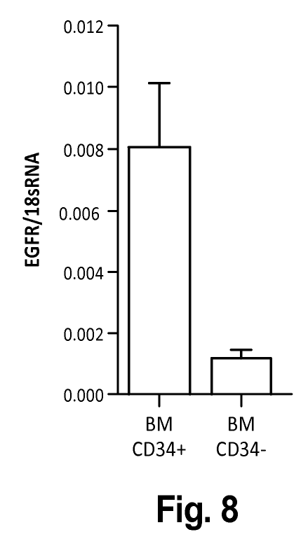

To investigate the relevance of altered Cdc42 activity for our genetic model of interstrain differences in mobilization efficiency, we determined the activity of Cdc42 in GCSF treated animals from control B6 (poor mobilizer, EGF sensitive) and congenic line 1804 (good mobilizer, EGF insensitive). Consistent with the data presented so far which implies a negative role of strongly elevated activation of cdc42 upon G-CSF treatment, levels of Cdc42 activity were significantly increased in G-CSF treated animals in LD-BM cells from B6 animals relative to control non-treated animals, while not being altered in 1804 animals upon G-CSF treatment (FIG. 7e). Mobilization of HSPCs is a dynamic and complex process, with multiple cellular and molecular pathways involved. Our data supports in summary a role for EGFR signaling in regulating mobilization efficiency cell intrinsically in part via regulating the level of Cdc42 activity upon G-CSF treatment. To the best of our knowledge, this is a novel role for EGFR signaling in hematopoiesis, and might further imply an active role for EGFR signaling in other aspects of hematopoiesis. In addition, our data suggests a role for Cdc42 in hematopoiesis. The expression of the EGFR is primarily regulated by the abundance of its mRNA, and the level of expression of the human EGFR gene correlates with allelic polymorphisms in the gene. Cytokine induced mobilization of HSPC's is evolutionarily conserved from mice to humans. A likely evolutionary mouse human conservation of EGFR pathway for regulating mobilization of human hematopoietic cells is further strongly supported by the fact that the EGFR is also expressed on primary human CD34+ hematopoietic progenitor cells. This is evidenced by FIG. 8, which shows that EGFR is expressed in human hematopoietic progenitor cells.

The evidence presented demonstrates that administering G-CSF with EGFR inhibitors enhances HSPC mobilization. Therefore, the method further contemplates that G-CSF/EGFR inhibitor combinations may be provided to the practitioners pre-formulated for virtually any means of co-administering. Further, the G-CSF/EGFR inhibitor combinations also can be included in kits, which may be suitable when the particular combination is used in a regimen where co-administration is not preferred or desired. Persons of ordinary skill in the art will appreciate the numerous ways in which G-CSF/EGFR inhibitor combinations may be provided to practitioners, hospitals, pharmacies and the like.

What is claimed is:

1. A combination of therapeutic agents comprising;
an amount of G-CSF (granulocyte colony-stimulating factor) effective to enhance mobilization of hematopoietic stem cells and progenitor cells (HSPC); and
an amount of at least one inhibitor of EGFR (epidermal growth factor receptor) effective to inhibit the effects of EGFR signaling.

2. The combination of claim 1, wherein the at least one inhibitor of EGFR is selected from the group consisting of an antibody directed against EGFR, an analogue of EGF (epidermal growth factor), a tyrosine kinase inhibitor and a Cdc42 inhibitor.

3. The combination of claim 2, wherein the at least one inhibitor of EGFR is a tyrosine kinase inhibitor.

4. The combination of claim 3, wherein the tyrosine kinase inhibitor is erlotinib.

5. The combination of claim 3, wherein the tyrosine kinase inhibitor is gefitinib.

6. The combination of claim 3, wherein the rho-GTPase inhibitor inhibits cdc42.

7. A combination of therapeutic agents comprising;
an effective amount of G-CSF (granulocyte colony-stimulating factor); and
an effective amount of at least one inhibitor that inhibits at least one event resulting from EGFR (epidermal growth factor receptor) activation.

8. The combination of claim 7, wherein the inhibitor is an antibody that prevents EGF binding by EGFR.

9. The combination of claim 8, wherein the inhibitor is Erbitux.

10. The combination of claim 7, wherein the inhibitor reduces the activity of the EGFR protein kinase activity relative to the EGFR protein kinase activity in the absence of the inhibitor.

11. The combination of claim 10, wherein the inhibitor is either erlotinib or gefitinib.

12. The combination of claim 7, wherein the at least one event is selected from the group consisting of ligand binding to EGFR, activating the EGFR tyrosine kinase and increasing the cellular level of Cdc42 activity.

13. The combination of claim 8, wherein the at least one event inhibited is the formation of Cdc42 activity.

14. The combination of claim 8, wherein the at least one event is EGF binding to the EGFR.

15. A method of enhancing G-CSF-induced mobilization of hematopoietic stem and progenitor cells in a subject in need thereof, the method comprising administering a therapeutic combination comprising;
an amount of G-CSF (granulocyte colony-stimulating factor) effective to enhance mobilization of hematopoietic stem cells and progenitor cells (HSPC); and
an amount of at least one inhibitor of EGFR (epidermal growth factor receptor) effective to inhibit EGFR signaling.

16. An improved regimen for mobilizing hematopoietic stem and progenitor cells in a subject in need thereof, the regimen comprising;
an amount of G-CSF (granulocyte colony-stimulating factor) effective to enhance mobilization of hematopoietic stem cells and progenitor cells (HSPC);
an amount of at least one inhibitor of EGFR (epidermal growth factor receptor) effective to inhibit EGFR signaling; and
wherein the at least one inhibitor of EGFR signaling is selected from the group consisting of Erbitux, erlotinib, gefitinib.

17. A combination of therapeutic agents comprising;
an amount of AMD3100 effective to enhance mobilization of hematopoietic stem cells and progenitor cells (HSPC); and
an amount of at least one inhibitor of EGFR (epidermal growth factor receptor) effective to inhibit the effects of EGFR signaling.

18. The combination of claim 17, wherein the at least one inhibitor of EGFR is selected from the group consisting of an antibody directed against EGFR, an analogue of EGF (epidermal growth factor), a tyrosine kinase inhibitor and a Cdc42 inhibitor.

19. The combination of claim 18, wherein the at least one inhibitor of EGFR is a tyrosine kinase inhibitor.

20. The combination of claim 19, wherein the tyrosine kinase inhibitor is erlotinib.

21. A method of enhancing G-CSF-induced mobilization of hematopoietic stem and progenitor cells in a subject in need thereof, the method comprising administering a therapeutic combination comprising;
an amount of AMD3100 effective to enhance mobilization of hematopoietic stem cells and progenitor cells (HSPC); and
an amount of at least one inhibitor of EGFR (epidermal growth factor receptor) effective to inhibit EGFR signaling.

22. The combination of claim 21, wherein the at least one inhibitor of EGFR is selected from the group consisting of an antibody directed against EGFR, an analogue of EGF (epidermal growth factor), a tyrosine kinase inhibitor and a Cdc42 inhibitor.

23. The combination of claim 22, wherein the at least one inhibitor of EGFR is a tyrosine kinase inhibitor.

24. The combination of claim 23, wherein the tyrosine kinase inhibitor is erlotinib.
